(19) United States
(12) Patent Application Publication
(10) Pub. No.: US 2006/0292222 A1
(43) Pub. Date: Dec. 28, 2006

(54) Drug delivery device having zero or near zero-order release kinetics

(76) Inventor: Matthew Jonasse, Sodus, NY (US)

Correspondence Address: Bausch & Lomb Incorporated, One Bausch & Lomb Place, Rochester, NY, 14604-2701, US

(21) Appl. No.: 11/472,237

(22) Filed: Jun. 21, 2006

Related U.S. Application Data
(60) Provisional application No. 60/692,352, filed on Jun. 21, 2005.

(51) Int. Cl. A61K 9/22

(52) U.S. Cl. 424/468; 604/890.1

(57) ABSTRACT

The present invention is directed to an improved sustained release drug delivery device comprising a drug core, a cup, and a prefabricated crystalline or semi-crystalline polymeric permeable plug.

### CROSS REFERENCE

This application claims the benefit of Provisional Patent Application No. 60/692,352 filed Jun. 21, 2005 and is incorporated herein by reference.

### FIELD OF THE INVENTION

The present invention relates to improved delivery devices and methods of use. More particularly, the present invention relates to minimally invasive sustained release delivery devices particularly suitable for the delivery of therapeutic agents to limited access regions, such as the posterior chamber of the eye. Devices of the invention herein demonstrate zero order or near-zero order release kinetics without an initial spike of drug release.

### BACKGROUND

Over the years, various drugs have been developed to assist in the treatment of a wide variety of ailments and diseases. However, in many instances such drugs are not capable of being administered either orally or intravenously without the risk of various detrimental side effects.

The delivery of drugs to the eye presents many challenges. The ocular absorption of systemically administered pharmacologic agents is limited by the blood ocular barrier, namely the tight junctions of the retinal pigment epithelium and vascular endothelial cells. High systemic doses can penetrate this blood ocular barrier in relatively small amounts, but expose the patient to the risk of systemic toxicity. Topical delivery of drugs often results in limited ocular absorption due to the complex hydrophobic/hydrophilic properties of the cornea and sclera. Additionally, topical agents are mechanically removed by the blink mechanism such that only approximately 15% of a single drop is absorbed. Diffusion of topically administered drugs to the posterior chamber occurs, but often at sub-therapeutic levels. Intravitreal injection of drugs is an effective means of delivering a drug to the posterior segment in high concentrations. However, these repeated intraocular injections carry the risk of infection, hemorrhage and retinal detachment. Patients also find this procedure somewhat difficult to endure.

While intraocular devices exist which allow delivery of therapeutic agents to the eye, a need still remains for a device which accomplishes controlled, sustained delivery to a specific region of the eye, is implantable and removable without requiring long full thickness scleral incisions, does not cause undue patient irritation or discomfort, is stable within the specific region of the eye and is capable of delivering a wide range of small molecule, gene and protein therapeutics.

The need for a better release system is especially significant in the treatment of eye diseases that require the use of steroids. An initial burst effect of the steroid may contribute to undesirable side effects such as increased intraocular pressure or the formation of cataracts. Thus, there remains a long-felt need in the art for an improved device for providing sustained release of a drug to a patient to obtain a desired local or systemic physiological or pharmacological effect without demonstrating an initial burst effect.

### SUMMARY OF THE INVENTION

The sustained release drug delivery device according to the first embodiment of the present invention comprises:

a) a drug core comprising a therapeutically effective amount of at least one agent effective in obtaining a diagnostic effect or effective in obtaining a desired local or systemic physiological or pharmacological effect;

b) a cup essentially impermeable to the passage of the agent that surrounds and defines an internal compartment to accept the drug core, the cup comprising an open top end with at least one recessed groove around at least some portion of the open top end of the cup; and

c) a prefabricated plug which is permeable to the passage of the agent, the prefabricated plug comprising at least one crystalline or semi-crystalline polymer, the prefabricated plug is positioned at the open top end of the cup wherein the groove interacts with the prefabricated plug holding it in position and closing the open top end, the prefabricated plug allowing passage of the agent out of the drug core, through the prefabricated plug, and out the open top end of the cup; wherein the thickness and degree of crystallinity of the plug results in zero order or near zero order release kinetics for at least 120 days.

In accordance with another embodiment of the present invention is a sustained release drug delivery device comprising:

a) a drug core comprising at least one agent effective in obtaining a diagnostic effect or effective in obtaining a desired local or systemic physiological or pharmacological effect;

b) a cup essentially impermeable to the passage of the agent that surrounds and defines an internal compartment to accept the drug core, the cup comprising an open top end and at least one lip around at least a portion of the open top end of the cup; and

c) a prefabricated plug permeable to the passage of the agent positioned at the open top end of the cup, the prefabricated plug comprising at least one crystalline or semi-crystalline polymer, wherein the lip interacts with the prefabricated plug holding it in position and closing the open top end, the permeable plug allowing passage of the agent out of the drug core, through the permeable plug, and out the open top end of the cup at zero order or near zero order release kinetics for at least 120 days.

This invention is also directed to a method for providing controlled and sustained administration of an agent effective in obtaining a desired local or systemic physiological or pharmacological effect comprising inserting in a desired location in the body of a mammalian organism sustained release drug delivery devices of the first and second embodiments of the present invention.

A method of manufacture of a sustained release drug delivery device according to the present invention comprises:

a) manufacturing a drug core comprising at least one agent effective in obtaining a diagnostic effect or effective in obtaining a desired local or systemic physiological or pharmacological effect;

b) providing a cup essentially impermeable to the passage of the agent that surrounds and defines an internal compartment to accept the drug core, the cup comprising an open top end with at least one recessed groove around at least some portion of the open top end of the cup;

c) inserting the drug core into the cup; and

d) placing a prefabricated plug which is permeable to the passage of the agent into the open top end of the cup, the prefabricated plug comprising at least one crystalline or semi-crystalline polymer, wherein the groove interacts with the permeable member holding it in position and closing the open top end, the permeable plug allowing passage of the agent out of the drug core, through the permeable plug, and out the open top end of the cup at zero order or near zero order release kinetics for at least 120 days.

The present invention is further directed to a method of manufacturing a sustained release drug delivery device comprising:

a) manufacturing a drug core comprising at least one agent effective in obtaining a diagnostic effect or effective in obtaining a desired local or systemic physiological or pharmacological effect;

b) providing a cup essentially impermeable to the passage of the agent that surrounds and defines an internal compartment to accept the drug core, the cup comprising an open top end with at least one lip extending around at least a portion of the open top end of the cup;

c) inserting the drug core into the cup; and

d) placing a prefabricated plug comprising at least one crystalline or semi-crystalline polymer which is permeable to the passage of the agent into the open top end of the cup wherein the lip interacts with the permeable member holding it in position and closing the open top end, the permeable plug allowing passage of the agent out of the drug core, through the permeable plug, and out the open top end of the cup at zero order or near zero order release kinetics for at least 120 days.

### BRIEF DESCRIPTION OF THE DRAWINGS

The drawings, which are not drawn to scale, are set forth to illustrate various embodiments of the invention. The drawings are as follows:

FIG. 1 of the present invention is an enlarged cross-sectional view down the center of one embodiment of the sustained release drug delivery device showing a cup with a lip extending inward around some portion of the open end of the cup, the cup acting as a reservoir for an agent, and a prefabricated permeable plug.

FIG. 2 of the present invention is an enlarged cross-sectional view down the center of another embodiment of the sustained release drug delivery device showing a cup with a recessed groove around some portion of the inside of the open end of the cup, the cup acting as a reservoir for an agent, and a prefabricated permeable plug.

FIG. 3 of the present invention is an enlarged top view of another embodiment of the sustained release drug delivery device showing a cup with a plurality of lips extending inward around at least a portion of the open end of the cup, the cup acting as a reservoir for an agent, and a prefabricated permeable plug.

FIG. 4 of the present invention is an enlarged cross-sectional view down the center of another embodiment of the sustained release drug delivery device showing a cup with a plurality of lips and a suture tab, the cup acting as a reservoir for an agent, and a prefabricated permeable plug.

FIG. 5 is an enlarged top view of the embodiment of the sustained release drug delivery device in FIG. 1 showing a lip extending outward around only a portion of the open top end of the cup.

FIG. 6 is a graphical representation of the release rate of a device of the present invention as compared to a prior art device.

FIG. 7 is a graphical representation of the zero order or near zero order release kinetics of a device according to the invention herein.

### DETAILED DESCRIPTION OF THE INVENTION

The inventor has unexpectedly discovered a sustained release drug delivery device design that is structurally stable and provides zero order or near zero order release kinetics without an initial burst effect such as is found in current designs that are known in the art. The present invention also allows for commercial manufacture.

In one embodiment, the device includes an impermeable cup made of silicone, the cup acts as a reservoir for a drug core containing an agent such as fluocinolone acetonide. The open end of the cup has lips extending inwardly around a portion of the top open end of the cup. A prefabricated permeable crystalline or semi-crystalline plug is placed in the recess between the drug core and the lips wherein the lips interact with the plug holding it in position and closing the open top end. Together the cup with lips and the prefabricated permeable plug act as a reservoir surrounding the drug core and keeping it in place.

The expression “agent” as used herein broadly includes any compound, composition of matter, or mixture thereof that can be delivered from the device to produce a beneficial and useful result.

The term “impermeable” refers to a material that is sufficiently impermeable to environmental fluids as well as ingredients contained within the delivery device, such that the migration of such fluids and ingredients into or out of the device through the impermeable material is so low as to have substantially no adverse impact on the function of the device.

The term “permeable” refers to a material that is capable of being passed through or permeated. Permeating includes passing through openings, holes, pores, or intersections that result from the crystallinity of the material.

The term “semi-crystalline” refers to polymers that display a glass transition temperature and melting transition character on differential scanning calorimetry (DSC) indicative of semi-crystalline polymeric materials. Additional testing such as stress-strain curves may be used to differentiate semi-crystalline polymeric materials from covalently cross-linked polymeric materials. Further information regarding such materials can be found in Provisional Patent application No. 60/692,664, filed on even date herewith under Express Mail No. ED278098135US, the contents of which are incorporated herein by reference.

The term “drug core” refers to any drug supply, drug depot, drug in suspension, reservoir or drug matrix. It includes one or more agents necessary to obtain the desired diagnostic effect or local or systemic physiological or pharmacological effect. It includes any excipients, suspending agents, or binders. Reference may be made to any standard pharmaceutical textbook such as Remington's Pharmaceutical Sciences. The drug core can be in liquid form, solid form, in dispersion, or any other form known in the art. Solid dose includes all conventional solid dose forms known in the art including tablets and pellets. Dispersions include all conventional forms known in the art, such as liquid in liquid dispersions and solid in liquid dispersions.

The expression “passageway” as used herein includes an aperture, orifice, or bore sufficient to allow the agent to pass through. The passageway can be formed by mechanical procedures such as erosion, laser, or molding; and chemical procedures.

The expression “zero order or near zero order” as applied to release kinetics of drug delivery devices according to the invention herein is intended to include devices whose release rate is within ±0.0050 ug/day, preferably within ±0.0025 ug/day, more preferably within ±0.0015 ug/day throughout the release period up to at least 120 days. Referring to the drawing figures, like reference numerals designate identical or corresponding elements throughout the several figures.

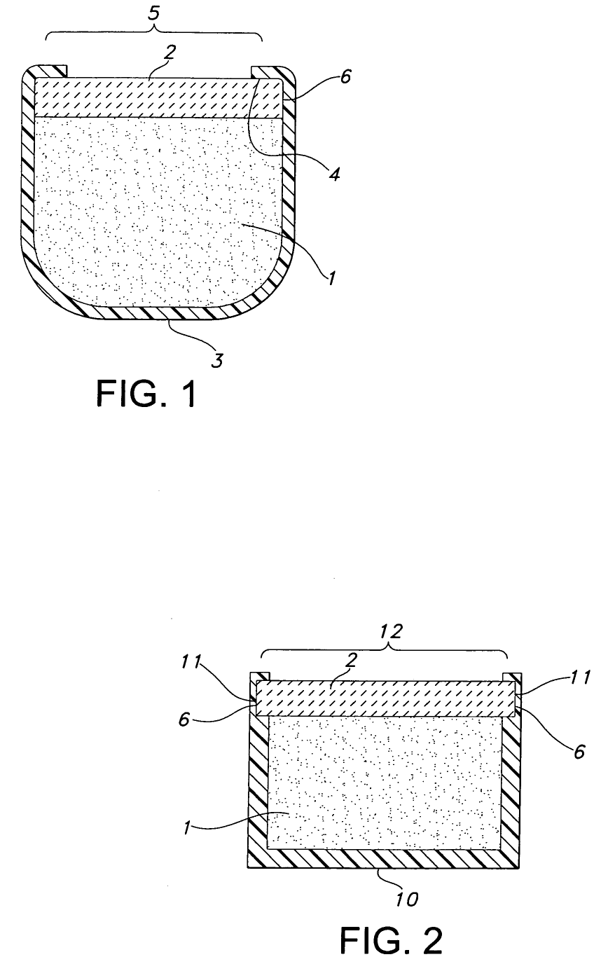

Turning now to the drawings in detail, which examples are not to be construed as limiting, one embodiment of a device is indicated in FIG. 1. While the device shown in FIG. 1 is generally U like in shape, the cup can be any open container or bowl of any shape. FIG. 1 is a cross sectional view of a drug delivery device in accordance with the present invention. FIG. 1 includes an impermeable cup 3 containing a drug core 1 comprising an agent, the cup 3 has lips 4 extending inward around the open top end 5 of the cup 3; and a prefabricated plug 2 formed of a material permeable to the passage of agent contained in the drug core 1. The prefabricated plug 2 is positioned in the recess between the top of the drug core 1 and below the lips 4 such that the lips 4 interact with the prefabricated plug 2 holding it in position and closing the open top end 5 of the cup 3.

The lips 4 are the same impermeable material as the cup 3 and protrude inwardly from the top open end 5 of the cup 3. The cup 3 and lips 4 are formed in a single design to provide structural integrity to the device and facilitate manufacturing and handling. The lips 4 are designed to enable the prefabricated plug 2 to be easily put into place and then to hold the plug 2 in place during use. They can vary in size or shape. The lips 4 of the present invention include nubs, tabs, ridges, and any other raised or protruding member.

By prefabricating the permeable plug 2 it can be put into or securely placed in the device in one step. The prefabricated plug 2 can be fabricated or machined to various dimensional specifications which can be used to control diffusion properties to achieve a desired release rate. The same cup and lips design can be used for implants with a variety of release rates making it possible to use a single manufacturing line or type of equipment. Thus, the present invention allows for ease of construction by more standard manufacturing techniques into devices with different release rates. Materials useful for the prefabricated permeable plug are crystalline or semi-crystalline polymeric materials that are not covalently cross linked. Such materials maintain their rigidity and display their permeability characteristics based upon the degree of crystallinity and the thickness of the prefabricated plug. The degree of crystallinity and thickness of the prefabricated plug can be varied to tailor the plug to the desired active to be released as well as the desired release profile. The thickness can vary depending upon the size of the device and its intended use. Thicknesses ranging about 75 μm to about 250 μm may be entirely suitable. For certain applications the thickness of the prefabricated permeable plug may be between about 100 μm and about 150 μm.

Together the cup 3 with lips 4 and the prefabricated permeable plug 2 act as a reservoir surrounding the drug core 1 and keeping it in place. The agent diffuses out of the drug core 1, through the prefabricated permeable plug 2, and out the open top end 5. The prefabricated plug 2 has substantially the same radial extent as the cup 3, so that the only diffusion pathway is out of the plug 2 and not around the sides 6. Glue, a polymeric substance, or other adhesion means can be employed to further bond the plug to the cup.

The invention further relates to a method for treating a mammalian organism to obtain a desired local or systemic physiological or pharmacological effect. The method includes administering the sustained release drug delivery device to the mammalian organism and allowing the agent effective in obtaining the desired local or systemic physiological or pharmacological effect to pass through the plug 2. The term “administering”, as used herein, means positioning, inserting, injecting, implanting, or any other means for exposing the device to a mammalian organism. The route of administration depends on a variety of factors including type of response or treatment, type of agent, and the site of administration. However, the method is to insert the device into the target organ. In ocular applications, more preferably through a surgical procedure followed by suturing the device in place.

FIG. 2 illustrates an enlarged cross sectional view down the center of a sustained release drug delivery device in accordance with the present invention. FIG. 2 includes an impermeable cup 10 containing a drug core 1 comprising an agent, the cup 10 has a recessed groove 11 around the inside of the open top end 12 of the cup 10; and a prefabricated permeable plug 2 formed of a material permeable to the passage of agent contained in the drug core 1. The prefabricated permeable plug 2 is positioned such that the groove 11 interacts with the prefabricated permeable plug 2 holding it in position and closing the open top end 12 of the cup 10.

Together the cup 10 with the groove 11 and the prefabricated permeable plug 2 act as a reservoir surrounding the drug core 1 and keeping it in place. The agent diffuses out of the drug core 1, through the prefabricated permeable plug 2, and out the open top end 12. The prefabricated plug 2 has substantially the same radial extent as the groove 11, so that the only diffusion pathway is out of the plug 2 and not around the sides 6. Glue or other adhesion means can be employed to further bond the plug to the cup.

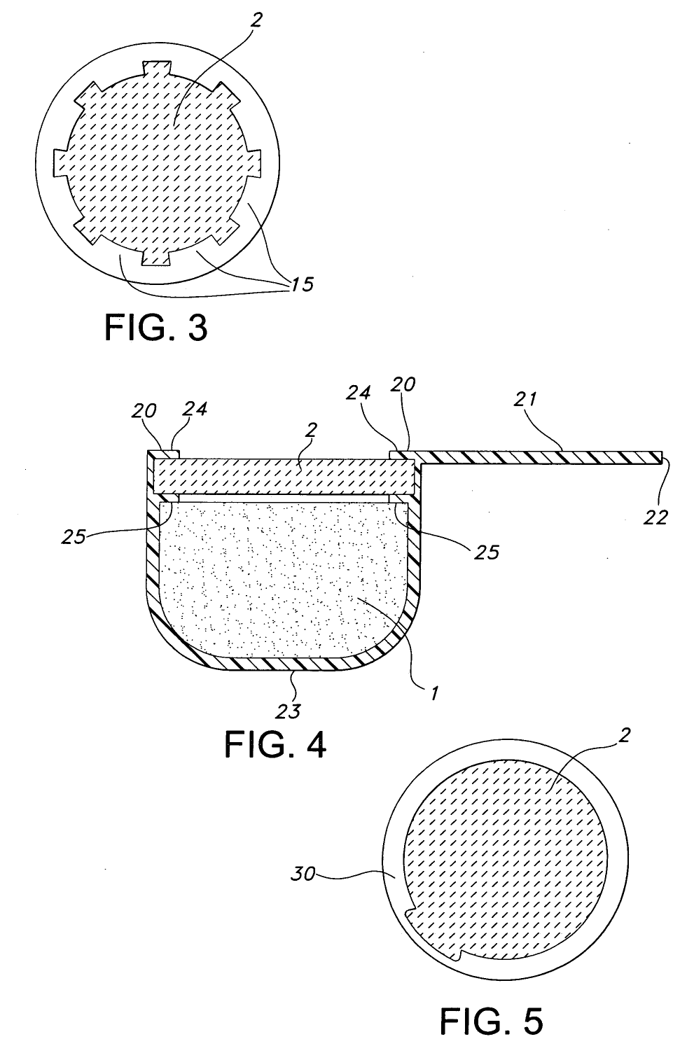

FIG. 3 is an enlarged top view of another exemplary embodiment of a sustained release drug delivery device of the present invention. The view in FIG. 3 is the top of a cup comprising a plurality of lips 15 extending inwardly around the open top end of the cup. The prefabricated permeable plug 2 is held in place by the lips 15 extending inwardly around the top open end of the cup. A plurality of lips 15 permits the prefabricated permeable plug to be put into the device more easily while still maintaining the integrity of the device.

FIG. 4 is an enlarged cross sectional view of a drug delivery device in accordance with the present invention. FIG. 4 includes an impermeable cup 23 containing a drug core 1 comprising an agent, the cup 23 has lips 24, 25 extending inward around the open top end 20 of the cup 23; and a prefabricated permeable plug 2 formed of a material permeable to the passage of agent contained in the drug core 1. The prefabricated permeable plug 2 is positioned in the recess between the first lip 25 and the second lip 24 such that the lips 24, 25 interact with the prefabricated permeable plug 2 holding it in position and closing the open top end 20 of the cup 23.

The cup 23 further comprises a suture tab 21 with a hole 22 through the proximal end through which a suture can be placed to anchor the device to a structure of the organism requiring treatment. The proximal end of the suture tab is at the point of attachment, i.e. the point where the suture is attached. The point of attachment is at the end of the suture tab opposite the cup.

The location of the suture and the structure the device is sutured to can be any that meet with current surgical techniques known in the art, such as the sclera of the eye. Depending upon the location of administration, the devices of the current invention may not require suturing in position.

Providing a suture hole 22 at the proximal end of the suture tab of the device enables the surgeon to attach the device without additional steps. Providing the suture hole reduces the possibility of tearing the tab while passing the needle through during surgery. Some materials, such as cured polyvinyl alcohol (PVA), are also very difficult to create a suture hole in once the device is assembled without causing cracks or breaks in the suture tab. Alternatively, the suture tab may comprise Dacron surgical fabric impregnated with a silicon containing polymer. In this embodiment (not shown) it may not be necessary to provide a suture hole 22.

The devices of the present invention may comprise a plurality of lips. These lips can be on the same vertical plane, as illustrated in FIG. 3, or on a different vertical plane, as illustrated in FIG. 4. The device may also be formed with any combination of lips in different vertical planes suitable to hold the prefabricated permeable plug in place. For example, a plurality of lips, as in FIG. 3, may be placed in the top vertical plane position 24 in FIG. 4) to facilitate be easily putping in the plug and a second single lip at a lower vertical plane (position 25 in FIG. 4) around the cup positioned above the drug core. The function of the lips is to hold the prefabricated permeable plug in place and prevent failure of the structural integrity of the device.

The devices of the present invention that employ recessed grooves to secure the prefabricated permeable plug in place may also have a plurality of grooves in the same or different vertical planes as described above.

FIG. 5 is an enlarged top view of another exemplary embodiment of a sustained release drug delivery device of the present invention. The view in FIG. 5 is the top of a cup comprising a single lip 30. The prefabricated permeable plug 2 is held in place by the lip 30 extending inwardly around the top open end of the cup. The single lip can extend around the entire diameter of the top open end of the cup or extend around some portion, as illustrated in FIG. 5.

In combination with the examples above, a variety of methods may also be utilized to provide adhesion of the prefabricated permeable plug to the cup portion of the device. These methods include the use of adhesives, polymers such as PVA, or any other procedure known in the art to provide adhesion at the points of contact between the prefabricated permeable plug and the cup. The methods to improve adhesion will vary depending on the materials that the components are manufactured from.

For example, the prefabricated permeable plugs or the cups of the present invention may also be treated before or after assembly with an adhesive, which would serve to further secure the prefabricated plug in the device. The sealant can be permeable or impermeable to the agent or agents in the device depending upon the method of application. For example, adhesives could be applied to only the edges of the plug and because the adhesive is present only on the edges, it improves the bond between the plug and the device without interfering with diffusion through the body of the prefabricated permeable plug. If the adhesive is permeable to the beneficial agent, such as in the case of a permeable polymer, it could be applied on top of the drug core or directly to the prefabricated permeable plug before the plug is put into place.

The prefabricated permeable plugs of the present invention should be sufficiently rigid to be easily put or inserted into the cup device. The entire plug can be made of a single material that is permeable to the agent(s) or some portion of the plug can be permeable. The amount of the plug that is permeable depends upon a number of characteristics including the desired release rate, the agent(s) used, and the duration of therapy needed. For example, a permeable material that is not sufficiently rigid to be easily put into place can be contained inside a holder of material that is sufficiently rigid. The holder should be designed to allow transport of the agent(s) through the permeable material, out the open top of the cup, and into the organism in need of such treatment. Sufficient surface area of the permeable material should be in contact with the area of treatment and the drug core such that it provides the desired therapeutic or diagnostic effect. The holder can be made from any material sufficiently rigid, such as metal, ceramics, glass, or polymers. For example, the holder can be formed as a rigid metal ring framing the edges of a disk of permeable material.

The lips of the cup in the present invention can extend either inwardly, as illustrated in the figures provided, or can extend outwardly from the top open end of the cup. The prefabricated plug would thus be formed as a cap, coming down over the edges of the cup and interacting with the outwardly extending lips. The prefabricated plug would be held in place by the interaction with the lips and close the open top end of the cup. Agent would move out of the drug core, through the open top end of the cup, and out through the prefabricated plug.

The grooves in the cup of the present invention can be placed on either the inside of the cup, as illustrated in the figure provided, or on the outside of the cup. The prefabricated plug would thus be formed as a cap, coming down over the edges of the cup and interacting with the grooves on the outside of the cup. The prefabricated plug would be held in place by the interaction with the grooves and close the open top end of the cup. Agent would move out of the drug core, through the open top end of the cup, and out through the prefabricated plug.

The drug core or reservoir contains an agent effective in obtaining a desired local or systemic physiological or pharmacological effect. The following classes of agents could be incorporated into the devices of the present invention: anesthetics and pain killing agents such as lidocaine and related compounds and benzodiazepam and related compounds; anti-cancer agents such as 5-fluorouracil, adriamycin and related compounds; anti-fungal agents such as fluconazole and related compounds; anti-viral agents such as trisodium phosphomonoformate, trifluorothymidine, acyclovir, ganciclovir, DDI and AZT; cell transport/mobility impending agents such as colchicine, vincristine, cytochalasin B and related compounds; antiglaucoma drugs such as beta-blockers: timolol, betaxolol, atenalol, etc; antihypertensives; decongestants such as phenylephrine, naphazoline, and tetrahydrazoline; immunological response modifiers such as muramyl dipeptide and related compounds; peptides and proteins such as cyclosporin, insulin, growth hormones, insulin related growth factor, heat shock proteins and related compounds; steroidal compounds such as dexamethasone, prednisolone and related compounds; low solubility steroids such as fluocinolone acetonide and related compounds; carbonic anhydrize inhibitors; diagnostic agents; antiapoptosis agents; gene therapy agents; sequestering agents; reductants such as glutathione; antipermeability agents; antisense compounds; antiproliferative agents; antibody conjugates; antidepressants; bloodflow enhancers; antiasthmatic drugs; antiparasiticagents; non-steroidal anti inflammatory agents such as ibuprofen; nutrients and vitamins; enzyme inhibitors; antioxidants; anticataract drugs; aldose reductase inhibitors; cytoprotectants; cytokines, cytokine inhibitors and cytokine protectants; uv blockers; mast cell stabilizers; and anti neovascular agents such as antiangiogenic agents like matrix metalloprotease inhibitors.

Examples of such agents also include neuroprotectants such as nimodipine and related compounds; antibiotics such as tetracycline, chlortetracycline, bacitracin, neomycin, polymyxin, gramicidin, oxytetracycline, chloramphenicol, gentamycin, and erythromycin; antiinfectives; antibacterials such as sulfonamides, sulfacetamide, sulfamethizole, sulfisoxazole; nitrofurazone, and sodium propionate; antiallergenics such as antazoline, methapyriline, chlorpheniramine, pyrilamine and prophenpyridamine; anti-inflammatories such as hydrocortisone, hydrocortisone acetate, dexamethasone 21-phosphate, fluocinolone, medrysone, methylprednisolone, prednisolone 21-phosphate, prednisolone acetate, fluoromethalone, betamethasone and triminolone; miotics and anti-cholinesterase such as pilocarpine, eserine salicylate, carbachol, di-isopropyl fluorophosphate, phospholine iodine, and demecarium bromide; mydriatics such as atropine sulfate, cyclopentolate, homatropine, scopolamine, tropicamide, eucatropine, and hydroxyamphetamine; sympathomimetics such as epinephrine; and prodrugs such as those described in Design of Prodrugs, edited by Hans Bundgaard, Elsevier Scientific Publishing Co., Amsterdam, 1985. In addition to the above agents, other agents suitable for treating, managing, or diagnosing conditions in a mammalian organism may be placed in the inner core and administered using the sustained release drug delivery devices of the current invention. Once again, reference may be made to any standard pharmaceutical textbook such as Remington's Pharmaceutical Sciences for the identity of other agents.

Any pharmaceutically acceptable form of such a compound may be employed in the practice of the present invention, i.e., the free base or a pharmaceutically acceptable salt or ester thereof. Pharmaceutically acceptable salts, for instance, include sulfate, lactate, acetate, stearate, hydrochloride, tartrate, maleate and the like.

A large number of polymers can be used to construct the devices of the present invention. The only requirements are that they are inert, non-immunogenic and of the desired permeability. Materials that may be suitable for fabricating the device include naturally occurring or synthetic materials that are biologically compatible with body fluids and body tissues, and essentially insoluble in the body fluids with which the material will come in contact. The use of rapidly dissolving materials or materials highly soluble in body fluids are to be avoided since dissolution of the wall would affect the constancy of the drug release, as well as the capability of the device to remain in place for a prolonged period of time.

Naturally occurring or synthetic materials that are biologically compatible with body fluids and eye tissues and essentially insoluble in body fluids which the material will come in contact include, but are not limited to, glass, metal, ceramics, polyvinyl acetate, cross-linked polyvinyl alcohol, cross-linked polyvinyl butyrate, ethylene ethylacrylate copolymer, polyethyl hexylacrylate, polyvinyl chloride, polyvinyl acetals, plasiticized ethylene vinylacetate copolymer, polyvinyl alcohol, polyvinyl acetate, ethylene vinylchloride copolymer, polyvinyl esters, polyvinylbutyrate, polyvinylformal, polyamides, polymethylmethacrylate, polybutylmethacrylate, plasticized polyvinyl chloride, plasticized nylon, plasticized soft nylon, plasticized polyethylene terephthalate, natural rubber, polyisoprene, polyisobutylene, polybutadiene, polyethylene, polytetrafluoroethylene, polyvinylidene chloride, polyacrylonitrile, cross-linked polyvinylpyrrolidone, polytrifluorochloroethylene, chlorinated polyethylene, poly(1,4′-isopropylidene diphenylene carbonate), vinylidene chloride, acrylonitrile copolymer, vinyl chloride-diethyl fumerale copolymer, butadiene/styrene copolymers, silicone rubbers, especially the medical grade polydimethylsiloxanes, ethylene-propylene rubber, silicone-carbonate copolymers, vinylidene chloride-vinyl chloride copolymer, vinyl chloride-acrylonitrile copolymer and vinylidene chloride-acrylonitride copolymer.

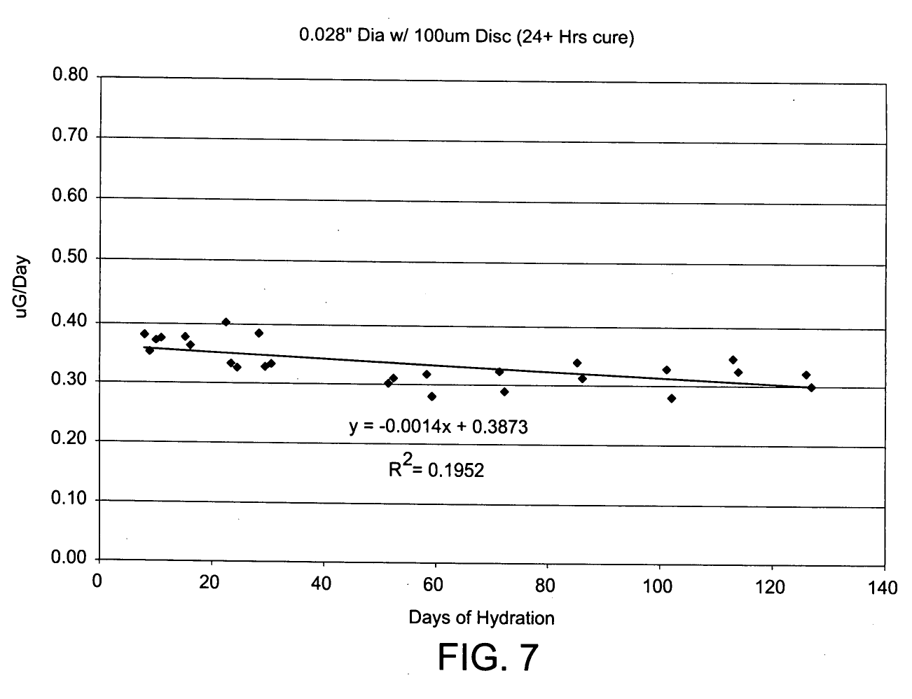

Additionally, the permeable member of the present invention can also be formed of any crystalline of semi-crystalline polymeric materials which will allow diffusion of the agent out of the drug core through the cup open end in a zero order or near zero order release rate. In certain embodiments it is possible to maintain this release rate for an extended period of time. For example, for at least 30, 60, 90, or 120 days or more (FIG. 7). If the prefabricated plug is made entirely from the permeable material, the material should be sufficiently rigid under the conditions of use to hold its shape and seal the top open end of the cup. Such materials include, but are not limited to, crystalline or semi-crystalline polymeric materials such as poly vinyl alcohol, polyethylene or polypropylene.

The device can be formulated in any convenient shape. For example, the device can be of any geometric shape dimensionally suitable for insertion in the eye. Thus, the device can be ellipsoid, rectangular, round, etc.

The dimensions of the device can vary with the size of the device, the size of the core or reservoir, and the membrane that surrounds the core or reservoir. The physical size of the device should be selected so that it does not interfere with physiological functions at the implantation site of the mammalian organism. The targeted disease states, type of mammalian organism, location of administration, and agents or agent administered are among the factors which would affect the desired size of the sustained release drug delivery device.

The devices according to the present invention may be made in a variety of ways. For example, if the cup is going to be made entirely of polymer, then the polymer can be injection molded or die cast into a desired shape and size. The prefabricated permeable plug can be formed in a plug of the desired dimensions by molding, machining, stamping, or any other conventional means depending on the materials selected. The agent can be filled into the reservoir by any conventional means such as syringe or pipette. The agent can also be made as a solid dose form such as a tablet or pellet and placed into the reservoir.

The preceding descriptions of how to make the devices of the present invention is merely illustrative and should not be considered as limiting the scope of the invention in any way. In particular, the methods of making the device depend on the identity of the agent.

The devices may be surgically implanted at or near the site of action. This is the case for devices of the present invention used in treatment of ocular conditions, primary tumors, rheumatic and arthritic conditions, and chronic pain. The devices may also be implanted subcutaneously, intramuscularly, intraarterially, or intraperitoneally. This is the case when devices are to give sustained systematic levels and avoid premature metabolism. In addition, such devices may be administered orally.

Once in place, the device functions as a drug reservoir gradually releasing drug to the organ such as the eye and surrounding tissue. This device is particularly useful for treating ocular conditions such as glaucoma, proliferative vitreoretinopathy, diabetic retinopathy, uveitis, and keratitis.

As would be readily understood by one skilled in the art, the amounts, materials, and dimensions depend on the method of administration, the effective agent used, the polymers used, the desired release rate and the like. Likewise, actual release rates and release duration depend on a variety of factors in addition to the above, such as the disease state being treated, the age and condition of the patient, the route of administration, as well as other factors which would be readily apparent to those skilled in the art.

Thus, the devices of the present invention provide many important advantages over previously known sustained release drug delivery devices. The cup and prefabricated plug design of the present invention provide an improved device that maintains its physical and chemical integrity in both the environments of use and in the presence of agent during the controlled and continuous dispensing of agent over a prolonged period of time.

Because of the structural integrity of the present design, the need for coatings and multiple layers can be eliminated. For transport of agent out of the device and into the target area, it is only necessary that the permeable layer cover the portions of the device not covered with the impermeable layer. However, the permeable layer may be coextensive with portions of the device covered with the impermeable layer.

Another advantage of the devices of the present invention is the ease of construction by more standard manufacturing techniques into devices with different release rates. The plug can be made to various crystalline and thickness dimensional specifications that can be used to control diffusion properties to achieve a desired release rate. The same cup can be used for implants with different release rates making it possible to use a single manufacturing line or type of equipment.

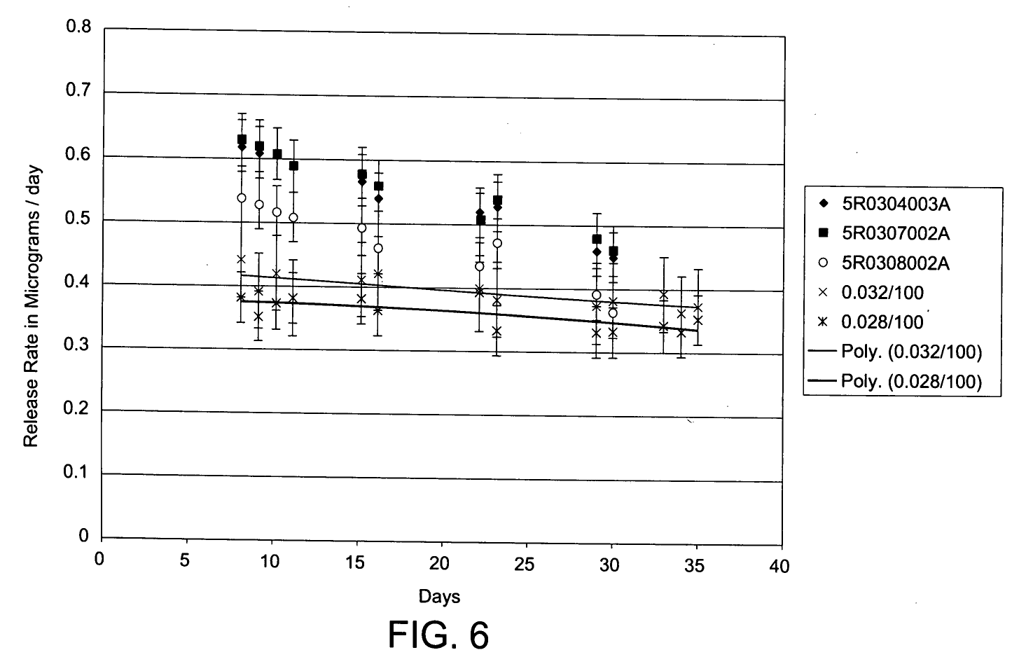

In addition, the use of a single cup and prefabricated permeable plug to form the container or drug reservoir of the present design provides more consistent and improved sealing capacity over the devices in the prior art. This permits the therapeutic program to be precisely controlled and the release of drug to be constant and predicted with accuracy (FIG. 6).

The following specific examples demonstrate a sustained release drug delivery device design of the present invention. However, it is to be understood that these example are for illustrative purposes only and do not purport to be wholly definitive as to the conditions and scope.

### EXAMPLE 1

A device according to the present invention is prepared. The cup is made of silicone and has eight inwardly extending lips around the top open end of the cup. The cup comprises a suture tab with a hole at the end of the tab opposite the cup. The drug core is formed as a pellet composed of a 2.5 mg core of fluocinolone acetonide and inserted into the cup. A plug of crystalline or semi-crystalline PVA is formed to the same radial extend as the cup. A few drops of PVA are placed on top of the drug core, and then the PVA plug is put into the recess between the top of the drug core and the lips. The lips thereby interacting with the plug and holding it in place.

### EXAMPLE 2

The device of example 1 above is measured for release rate by placing the dry implant into phosphate buffered saline under “sink” conditions at 37° C., where “sink” conditions are defined as less than 10% of the saturation concentration of fluocinolone acetonide. The implant is then allowed to hydrate for several days in order to achieve a steady state release rate. Once steady state release conditions are met, the buffer is changed daily, sampled and assayed for fluocinolone acetonide in order to determine a daily release rate. From the data that is collected, the release rate of the device can be determined.

From the foregoing description, one of ordinary skill in the art can easily ascertain the essential characteristics of the instant invention, and without departing from the spirit and scope thereof, can make various changes and/or modifications of the inventions to adapt it to various usages and conditions. As such, these changes and/or modifications are properly, equitably, and intended to be, within the full range of equivalence of the following claims.

What is claimed is:

1. A sustained release drug delivery device comprising:
a) a drug core comprising a therapeutically effective amount of at least one agent effective in obtaining a diagnostic effect or effective in obtaining a desired local or systemic physiological or pharmacological effect;
b) a cup essentially impermeable to the passage of the agent that surrounds and defines an internal compartment to accept the drug core, the cup comprising an open top end with at least one recessed groove around at least some portion of the open top end of the cup; and
c) a prefabricated plug which is permeable to the passage of the agent, the prefabricated plug comprising at least one crystalline or semi-crystalline polymer, the prefabricated plug is positioned at the open top end of the cup wherein the groove interacts with the prefabricated plug holding it in position and closing the open top end, the prefabricated plug allowing passage of the agent out of the drug core, through the prefabricated plug, and out the open top end of the cup; wherein the thickness and degree of crystallinity of the plug results in zero order or near zero order release kinetics for at least 120 days.

2. The sustained release drug delivery device according to claim 1, wherein the cup is made of a polymer, a metal, a ceramic, or glass.

3. The sustained release drug delivery device according to claim 1, wherein the cup further comprises a suture tab.

4. The sustained release drug delivery device according to claim 3, wherein the suture tab has a hole through the proximal end through which a suture can be placed to anchor the device to a structure.

5. The sustained release drug delivery device according to claim 3, wherein the cup is made of silicone.

6. The sustained release drug delivery device according to claim 1, wherein the prefabricated plug is between about 75 μm to about 250 μm in thickness.

7. The sustained release drug delivery device according to claim 1, wherein the cup further comprises a plurality of recessed grooves around at least some portion of the open top end of the cup.

8. The sustained release drug delivery device according to claim 1, wherein the agent is a low solubility agent.

9. The sustained release drug delivery device according to claim 1, wherein the agent is selected from a group consisting of immune response modifiers, neuroprotectants, corticosteroids, angiostatic steroids, anti-parasitic agents, antiglaucoma agents, antibiotics, anti-sense compounds, anti-angiogentic compounds, differentiation modulators, anti-viral agents, anti-cancer agents, and nonsteroidal anti-inflammatory agents.

10. The sustained release drug delivery device according to claim 1, wherein the drug core comprises a plurality of agents.

11. The sustained release drug delivery device according to claim 1, wherein the prefabricated plug comprises a holder and a permeable member.

12. A sustained release drug delivery device comprising:
a) a drug core comprising at least one agent effective in obtaining a diagnostic effect or effective in obtaining a desired local or systemic physiological or pharmacological effect;
b) a cup essentially impermeable to the passage of the agent that surrounds and defines an internal compartment to accept the drug core, the cup comprising an open top end and at least one lip around at least a portion of the open top end of the cup; and
c) a prefabricated plug permeable to the passage of the agent positioned at the open top end of the cup, the prefabricated plug comprising at least one crystalline or semi-crystalline polymer, wherein the lip interacts with the prefabricated plug holding it in position and closing the open top end, the permeable plug allowing passage of the agent out of the drug core, through the permeable plug, and out the open top end of the cup at zero order or near zero order release kinetics for at least 120 days.

13. The sustained release drug delivery device according to claim 12, wherein the lip extends around the entirety of the open top end of the cup.

14. The sustained release drug delivery device according to claim 12, wherein the cup comprises a plurality of lips at the open top end of the cup.

15. The sustained release drug delivery device according to claim 12, wherein the drug core comprises an effective amount of a low solubility agent.

16. The sustained release drug delivery device according to claim 12, wherein the agent is selected from a group consisting of immune response modifiers, neuroprotectants, corticosteroids, angiostatic steriods, anti-parasitic agents, anti-glaucoma agents, anti-biotics, anti-sense compounds, anti-angiogentic compounds, differentiation modulators, anti-viral agents, anti-cancer agents, and nonsteroidal anti-inflammatory agents.

17. The sustained release drug delivery device according to claim 12, wherein the cup is made of a polymer, a metal, a ceramic, or glass.

18. The sustained release drug delivery device according to claim 12, wherein the cup further comprises a suture tab.

19. The sustained release drug delivery device according to claim 18, wherein the cup is made of silicone.

20. The sustained release drug delivery device according to claim 12, wherein the prefabricated plug is between about 75 μm to about 250 μm in thickness.

21. The sustained release drug delivery device according to claim 18, wherein the suture tab has a hole through the proximal end through which a suture can be placed to anchor the device to a structure.

22. The sustained release drug delivery device according to claim 12, wherein the prefabricated plug comprises a holder and a permeable member.

23. The sustained release drug delivery device according to claim 12, wherein the drug core comprises a plurality of agents.

24. A method for providing controlled and sustained administration of an agent effective in obtaining a desired local or systemic physiological or pharmacological effect comprising:
inserting in a desired location in the body of a mammalian organism a sustained release drug delivery device comprising;
a) a drug core comprising a therapeutically effective amount of at least one agent effective in obtaining a diagnostic effect or effective in obtaining a desired local or systemic physiological or pharmacological effect;
b) a cup essentially impermeable to the passage of the agent that surrounds and defines an internal compartment to accept the drug core, the cup comprising an open top end with at least one recessed groove around at least some portion of the open top end of the cup; and
c) a prefabricated plug which is permeable to the passage of the agent, the prefabricated plug comprising at least one crystalline or semi-crystalline polymer, the prefabricated plug is positioned at the open top end of the cup wherein the groove interacts with the prefabricated plug holding it in position and closing the open top end, the prefabricated plug allowing passage of the agent out of the drug core, through the prefabricated plug, and out the open top end of the cup; wherein the thickness and degree of crystallinity of the plug results in zero order or near zero order release kinetics for at least 120 days.

25. The method according to claim 24, wherein the inserting step comprises inserting the sustained release drug delivery device in a location selected from a group consisting of the vitreous of the eye, under the retina, and onto the sclera.

26. The method according to claim 24, wherein the drug core comprises a plurality of agents.

27. The method according to claim 24, wherein the inserting step comprises injecting the sustained release drug delivery device at the desired location.

28. A method for providing controlled and sustained administration of an agent effective in obtaining a desired local or systemic physiological or pharmacological effect comprising:
inserting at a desired location in the body of a mammalian organism a sustained release drug delivery device comprising;
a) a drug core comprising at least one agent effective in obtaining a diagnostic effect or effective in obtaining a desired local or systemic physiological or pharmacological effect;
b) a cup essentially impermeable to the passage of the agent that surrounds and defines an internal compartment to accept the drug core, the cup comprising an open top end and at least one lip around at least a portion of the open top end of the cup; and
c) a prefabricated plug permeable to the passage of the agent positioned at the open top end of the cup, the prefabricated plug comprising at least one semi-crystalline polymer, wherein the lip interacts with the prefabricated plug holding it in position and closing the open top end, the permeable plug allowing passage of the agent out of the drug core, through the permeable plug, and out the open top end of the cup at zero order or near zero order release kinetics for at least 120 days.

29. The method according to claim 28, wherein the inserting step comprises inserting the sustained release drug delivery device in a location selected from a group consisting of the vitreous of the eye, under the retina, and onto the sclera.

30. The method according to claim 28, wherein the drug core contains a plurality of the agents.

31. The method according to claim 28, wherein the inserting step comprises injecting the sustained release drug delivery device at the desired location.

32. A method of manufacturing a sustained release drug delivery device comprising:
a) manufacturing a drug core comprising at least one agent effective in obtaining a diagnostic effect or effective in obtaining a desired local or systemic physiological or pharmacological effect;
b) providing a cup essentially impermeable to the passage of the agent that surrounds and defines an internal compartment to accept the drug core, the cup comprising an open top end with at least one recessed groove around at least some portion of the open top end of the cup;
c) inserting the drug core into the cup; and
d) placing a prefabricated plug which is permeable to the passage of the agent into the open top end of the cup, the prefabricated plug comprising at least one crystalline or semi-crystalline polymer, wherein the groove interacts with the permeable member holding it in position and closing the open top end, the permeable plug allowing passage of the agent out of the drug core, through the permeable plug, and out the open top end of the cup at zero order or near zero order release kinetics for at least 120 days.

33. The method of manufacturing a sustained release drug delivery device according to claim 32, wherein the drug core is manufactured as a solid dose form.

34. The method of manufacturing a sustained release drug delivery device according to claim 32, wherein the drug core is manufactured as a solid dispersion.

35. A method of manufacturing a sustained release drug delivery device comprising:
a) manufacturing a drug core comprising at least one agent effective in obtaining a diagnostic effect or effective in obtaining a desired local or systemic physiological or pharmacological effect;
b) providing a cup essentially impermeable to the passage of the agent that surrounds and defines an internal compartment to accept the drug core, the cup comprising an open top end with at least one lip extending around at least a portion of the open top end of the cup;
c) inserting the drug core into the cup; and
d) placing a prefabricated plug comprising at least one crystalline or semi-crystalline polymer which is permeable to the passage of the agent into the open top end of the cup wherein the lip interacts with the permeable member holding it in position and closing the open top end, the permeable plug allowing passage of the agent out of the drug core, through the permeable plug, and out the open top end of the cup at zero order or near zero order release kinetics for at least 120 days.

36. The method of manufacturing a sustained release drug delivery device according to claim 35, wherein the drug core is manufactured as a solid dose form.

37. The method of manufacturing a sustained release drug delivery device according to claim 35, wherein the drug core is manufactured as a solid dispersion.
